(19) United States
(12) Patent Application Publication
(10) Pub. No.: US 2013/0156728 A1
(43) Pub. Date: Jun. 20, 2013

(54) COMPOSITION FOR NEUROLOGICAL OR AUTOIMMUNE DISEASES

(71) Applicant: Daniel L. Kaufman, Los Angeles, CA (US)

(72) Inventor: Daniel L. Kaufman, Los Angeles, CA (US)

(73) Assignee: National Institutes of Health (NIH), U.S. Dept. of Health and Human Services (DHHS), U.S. Government, Bethesda, MD (US)

(21) Appl. No.: 13/699,605

(22) PCT Filed: May 26, 2011
(86) PCT No.: PCT/US2011/038243

§ 371 Date: Mar. 4, 2013

Related U.S. Application Data
(60) Provisional application No. 61/348,697, filed on May 26, 2010.

(51) Int. Cl. A61K 39/07; A61K 39/395; A61K 39/39

(52) U.S. Cl. CPC A61K 39/07; A61K 39/39; A61K 39/395
USPC 424/85.2; 424/246.1; 424/85.5; 424/130.1

(57) ABSTRACT

The present invention provides a composition effective for a neurological disease or an autoimmune disease and methods of making and using the same.

### CROSS-REFERENCE TO RELATED APPLICATIONS

This application claims the benefit of U.S. Provisional Patent Application No. 61/348,697, filed May 26, 2010, the teaching of which is incorporated herein by reference in its entirety.

### FEDERALLY SPONSORED RESEARCH AND DEVELOPMENT

This invention was made with Government support under Grant No. NS049121, awarded by the National Institutes of Health. The Government has certain rights in this invention.

### FIELD OF THE INVENTION

The present invention relates to a composition effective for a neurological disease or an autoimmune disease and methods of making and using the same.

### BACKGROUND OF THE INVENTION

Neurological and autoimmune diseases continue to poise major health threats in the population. Common neurological diseases include neurodegenerative diseases. Common autoimmune diseases include rheumatoid arthritis (RA) and type 1 diabetes (T1D). As example, a common neurodegenerative disorder is Parkinson's disease (PD). PD is a movement disorder that is chronic and progressive, meaning that symptoms continue and worsen over time. The cause is unknown, and although there is presently no cure, there are treatment options such as medication and surgery to manage its symptoms.

PD occurs when a group of cells in an area of the brain called the substantia nigra begin to malfunction and die. These cells in the substantia nigra produce a chemical called dopamine. Dopamine is a neurotransmitter, or chemical messenger, that sends information to the parts of the brain that control movement and coordination.

When a person has PD, their dopamine-producing cells begin to die and the amount of dopamine produced in the brain decreases. Messages from the brain telling the body how and when to move are therefore delivered more slowly, leaving a person incapable of initiating and controlling movements in a normal way.

A number of treatments can temporarily ameliorate PD symptoms, but none can slow the progressive loss of dopaminergic neurons. Likewise,

Therefore, there is a continuing need for a method and composition for treating PD.

Embodiments of the present invention address the above mentioned problems and needs.

### SUMMARY OF THE INVENTION

In one aspect, it is provided a composition. The composition can be a vaccine composition or a pharmaceutical composition. The composition comprises an effective amount of Bacillus Calmette-Guerin (BCG) and an optional second agent, wherein the composition is effective for inhibiting the progress of a neurological disease or an autoimmune disease or causing a neurological disease or an autoimmune disease to progress at a slower rate. In some embodiments, the composition is a neuroprotective vaccine. In some other embodiments, the composition is a neuroprotective drug.

The second agent can be a biologic compound, a polymer, or a small molecule. In some embodiments, the second agent is a therapeutic compound effective for Parkinson's disease (PD).

In some embodiments, the second agent is non-optional, and the second agent is an enzyme, a peptide, a compound or a combination of these.

In some embodiments, the second agent is non-optional, and the second agent is an enzyme, e.g., a glutamic acid decarboxylase (GAD). The GAD can be, for example, GAD65, or GAD67.

In some embodiments, the second agent is non-optional, and the second agent is an peptide, e.g., a GAD peptide.

In some embodiments, the second agent is non-optional, the second agent comprising an effective amount of an immunostimulatory factor that simulates a typical host response to BCG infection. In some embodiments, the immunostimulatory factor can be an inflammatory cytokine. In some embodiments, the immunostimulatory factor can be, e.g., IFNgamma, IL2β, IL-6, IL-32, IL-24 and combinations thereof.

In some embodiments, the second agent is non-optional, the second agent comprising an effective amount of a compound that simulates a BCG infection. In some embodiments, the second agent is a compound that binds to TLR-4 or TLR-2. In some embodiments, the second agent is one of agonists of receptors binding to TLR-4 or TLR-2 and antibodies that activate these receptors.

In some embodiments, the second agent is non-optional, the second agent comprising an effective amount of a compound that activates the signaling pathways used by TLR-4, TLR-2, IFNgamma, IL2β, IL-6, IL-32 and IL-24.

In some embodiments, the various embodiments of the composition of invention are vaccine compositions. In these embodiments, the composition can further comprise an adjuvant and/or an excipient.

In some embodiments, the various embodiments of the composition of invention are pharmaceutical compositions. In these embodiments, the composition comprises the BCG and the second agent. In these embodiments, the composition can comprise multiple formulations, e.g., one formulation comprising the BCG and another formulation comprising the second agent and further an excipient. In some embodiments, the excipient comprises a pharmaceutically acceptable carrier. In these embodiments, the formulation comprising the second agent can be a formulation for fast release or sustained release.

The composition can be formulated into formulations for systemic delivery or local delivery. In some embodiments, the composition can be in a formulation for oral delivery, inhalation, injection, implant, topical delivery, or transdermal delivery.

In another aspect, it is provided a method. The method comprises forming a composition of the various embodiments above.

In another aspect, it is provided a method of treating, preventing, or ameliorating a neurological disease or an autoimmune disease. The method comprises administering to a subject (a human being or an animal) a composition of the various embodiments disclosed herein. The neurological disease can be any neurological disease. In some embodiments, the neurological disease is a neurodegenerative disease, e.g., one of PD, Alzheimer's disease, and amyotrophic lateral sclerosis. The autoimmune disease can be any autoimmune disease. In some embodiments, the autoimmune disease is rheumatoid arthritis (RA) or type 1 diabetes (T1D).

### BRIEF DESCRIPTION OF THE DRAWINGS

FIG. 1 is characterization of a subacute MPTP model. (A) Four days after the last treatment, 20 mg/kg/day MPTP for 5 days decreased striatal dopamine level by 70% compared to PBS treated mice, while 10 mg/kg/day had no obvious effect on striatal dopamine levels. (B) Subacute MPTP treatment did not affect splenocyte counts of recipient mice, suggesting little/no immunotoxicity.

FIG. 2 shows that CFA and Copaxone®/CFA vaccination led to greater striatal DAT preservation and improved recovery. A) [³H]WIN-35,428 binding assays showed that both CFA and Copaxone®/CFA vaccinated animals had higher (but non-significant) DAT levels than non-vaccinated mice 4 days post MPTP. B) both CFA and Copaxone®/CFA vaccinated animals had higher DAT levels than non-vaccinated mice 21 days post MPTP (p<0.0001 by t-test for either group vs. MPTP control).

FIG. 3 shows that BCG conferred neuroprotection to vaccinated mice after subacute MPTP administration. A) Striatal tissues from BCG-vaccinated mice (n=18) showed 18% higher [3H]WIN-35,428 binding (p<0.01 by two-tailed t-test) than non-vaccinated MPTP-treated control mice (n=17). B) Striatal DA levels were 17% higher (p=0.01 by two-tailed t-test) in BCG-vaccinated mice (n=16) than in control mice (n=17).

FIG. 4 shows average SNc Iba-1+ cell number from BCG vaccinated mice is lower than that from MPTP control mice.

FIG. 5 shows the results of neuroprotective vaccination.

FIG. 6 shows the results of neuroprotective vaccination on WIN 35,428 binding.

### DETAILED DESCRIPTION OF THE INVENTION

In one aspect, it is provided a composition. The composition can be a vaccine composition or a pharmaceutical composition. The composition comprises an effective amount of Bacillus Calmette-Guerin (BCG) and an optional second agent, wherein the composition is effective for inhibiting the progress of a neurological disease or an autoimmune disease or causing a neurological disease or an autoimmune disease to progress at a slower rate. In some embodiments, the composition is a neuroprotective vaccine. In some other embodiments, the composition is a neuroprotective drug.

The second agent can be a biologic compound, a polymer, or a small molecule. In some embodiments, the second agent is a therapeutic compound effective for Parkinson's disease (PD).

In some embodiments, the second agent is non-optional, and the second agent is an enzyme, a peptide, a compound or a combination of these.

In some embodiments, the second agent is non-optional, and the second agent is an enzyme, e.g., a glutamic acid decarboxylase (GAD). The GAD can be, for example, GAD65, or GAD67.

In some embodiments, the second agent is non-optional, and the second agent is an peptide, e.g., a GAD peptide.

In some embodiments, the second agent is non-optional, the second agent comprising an effective amount of an immunostimulatory factor that simulates a typical host response to BCG infection. In some embodiments, the immunostimulatory factor can be an inflammatory cytokine. In some embodiments, the immunostimulatory factor can be, e.g., IFNgamma, IL2β, IL-6, IL-32, IL-24 and combinations thereof.

In some embodiments, the second agent is non-optional, the second agent comprising an effective amount of a compound that simulates a BCG infection. In some embodiments, the second agent is a compound that binds to TLR-4 or TLR-2. In some embodiments, the second agent is one of agonists of receptors binding to TLR-4 or TLR-2 and antibodies that activate these receptors.

In some embodiments, the second agent is non-optional, the second agent comprising an effective amount of a compound that activates the signaling pathways used by TLR-4, TLR-2, IFNgamma, IL2β, IL-6, IL-32 and IL-24.

In some embodiments, the various embodiments of the composition of invention are vaccine compositions. In these embodiments, the composition can further comprise an adjuvant and/or an excipient.

In some embodiments, the various embodiments of the composition of invention are pharmaceutical compositions. In these embodiments, the composition comprises the BCG and the second agent. In these embodiments, the composition can comprise multiple formulations, e.g., one formulation comprising the BCG and another formulation comprising the second agent and further an excipient. In some embodiments, the excipient comprises a pharmaceutically acceptable carrier. In these embodiments, the formulation comprising the second agent can be a formulation for fast release or sustained release.

The composition can be formulated into formulations for systemic delivery or local delivery. In some embodiments, the composition can be in a formulation for oral delivery, inhalation, injection, implant, topical delivery, or transdermal delivery.

In another aspect, it is provided a method. The method comprises forming a composition of the various embodiments above.

In another aspect, it is provided a method of treating, preventing, or ameliorating a neurological disease or an autoimmune disease. The method comprises administering to a subject (a human being or an animal) a composition of the various embodiments disclosed herein. The neurological disease can be any neurological disease. In some embodiments, the neurological disease is a neurodegenerative disease, e.g., one of PD, Alzheimer's disease, and amyotrophic lateral sclerosis. The autoimmune disease can be any autoimmune disease. In some embodiments, the autoimmune disease is rheumatoid arthritis (RA) or type 1 diabetes (T1D).

As used herein, the term “neurological disease” can be any disorder pertaining to the neurological system. In some embodiments, the neurological disease is a neurodegenerative disease. Examples of neurological disease can be, e.g., PD, Alzheimer's disease, and amyotrophic lateral sclerosis.

As used herein, the term Bacillus Calmette-Guerin (BCG) encompasses a wild-type BCG or a recombinant BCG (rBCG).

As used herein, the term “optional” shall mean having the options of being present or absent. When the term “optional” is removed, the option becomes being present (non-optional). For example, the term “optional second agent” shall mean that the second agent can be either present or absent. When the term “optional” is removed, the second agent has only one option, that is, being present (non-optional).

As used herein, the term “second agent” sometimes is used interchangeably with the term “other agent,” “another agent”, or “additional agent.”

As used herein, the term “fast release” shall mean burst release or release of substantially all BCG or the optional second agent within 60 minutes (e.g., within 30 minutes, with 10 minutes, within 5 minutes, or within 1 minute) after administration. Conversely, the term “sustained release” shall mean release of substantially all BCG or the optional second agent within a period from more than one hour to 1 day, from more than one hour to 1 week, from, from more than one hour to 2 weeks, from more than one hour to 30 days, from more than one hour to 2 months, from more than one hour to 6 months, or from more than one hour to 1 year.

As used herein, the term “neuroprotective” refers to an attribute(s) that causes a neurological disease to progress at a slower rate. In some embodiments, the term can refer to a reduction of the rate of progressive loss of dopaminergic neurons.

As used here, the term “slower rate” refers to a reduction of the progression rate of a disease or disorder (e.g., progressive loss of dopaminergic neurons, rate of rheumatoid arthritis progression or rate of T1D progression) by:

from about 5% to about 100%, e.g., about 5% to about 90%, about 5% to about 80%, 5% to about 70%, about 5% to about 60%, about 5% to about 50%, about 5% to about 40%, about 5% to about 30%, about 5% to about 20%, or about 5% to about 10%;

from about 10% to about 100%, e.g., about 10% to about 90%, about 10% to about 80%, about 10% to about 70%, about 10% to about 60%, about 10% to about 50%, about 10% to about 40%, about 10% to about 30%, or about 10% to about 20%;

from about 20% to about 100%, e.g., about 20% to about 90%, about 20% to about 80%, about 20% to about 70%, about 20% to about 60%, about 20% to about 50%, about 20% to about 40%, or about 20% to about 30%;

from about 30% to about 100%, e.g., about 30% to about 90%, about 30% to about 80%, about 30% to about 70%, about 30% to about 60%, about 30% to about 50%, or about 30% to about 40%;

from about 40% to about 100%, e.g., about 40% to about 90%, about 40% to about 80%, about 40% to about 70%, about 40% to about 60%, or about 40% to about 50%;

from about 50% to about 100%, e.g., about 50% to about 90%, about 50% to about 80%, about 50% to about 70%, or about 50% to about 60%;

from about 60% to about 100%, e.g., about 60% to about 90%, about 60% to about 80%, or about 60% to about 70%;

from about 70% to about 100%, e.g., about 70% to about 90%, or about 70% to about 80%;

from about 80% to about 100%, e.g., about 80% to about 90%; or

from about 90% to about 100%.

As used herein, the term “ligand” generally refers to a substance that forms a complex with a biomolecule to serve a biological purpose. In a narrower sense, it is a signal triggering molecule, binding to a site on a target protein.

As used herein, the term “adjuvant” refers to is a pharmacological or immunological agent that modifies the effect of other agents (e.g., drugs, vaccines) while having few if any direct effects when given by itself. Adjuvant can be an immunological adjuvant or can act as a stabilizing agent. An immunologic adjuvant can act in various ways in presenting an antigen to the immune system. Adjuvants can act as a depot for the antigen presenting the antigen over a long period of time, thus maximizing the immune response before the body clears the antigen. Examples of depot type adjuvants are oil emulsions. Adjuvants can also act as an irritant which causes the body to recruit and amplify immune response. Aduvants can also act as substances that can aid in stabilizing formulations of antigens.

As used herein, the term “excipient” can be used interchangeably with the term “carrier,” which is further described below.

BCG Vaccines for Neurological Disease and Autoimmune Diseases

A growing number of studies have shown that vaccination with CNS proteins can induce immune responses that inhibit neuronal degeneration in neuronal injury and neurodegeneration.

It is thought that oxidative stress; activated microglia and protein nitration contribute to loss of dopaminergic function in human PD. All of these potentially pathogenic factors are elicited by MPTP treatment. Therefore MPTP provides a model to test whether vaccine-induced immune responses can mitigate these factors. Using MPTP model, we tested neuroprotective vaccines and observed that complete Freund's adjuvant (CFA) treatment (alone) had a beneficial effect on dopaminergic markers (data not shown). However, CFA is not suitable for use in humans. The Bacillus Calmette-Guerin (BCG) vaccine that is used clinically against childhood tuberculosis and meningitis contains Mycobacterium bovis that is closely related to the Mycobacterium tuberculosis that is in CFA. We observed that a BCG vaccine was neuroprotective (data not shown). We believe that BCG vaccination induces a cytokine wave, some of which enters the CNS, and exterts a neuroprotective effect, perhaps by altering the phenotype of microglia.

While neuroprotective vaccines cannot correct basic intrinsic neuronal deficits, they may alter the environment to be more neurosupportive and reduce factors, either endogenous or exogenous, that promote neuronal dysfunction and death.

During PD pathogenesis, dopaminergic neuron loss occurs at a slow rate late in life. This degeneration is likely to result from the summed effects of toxic factors that were not sufficiently balanced by counteractive mechanisms. Lymphocytes do not significantly respond to the slow damage occurring over the course of human PD. However, vaccine-induced cytokines/chemokines and T cell responses can enter the CNS. They may alter the microenvironment in damaged areas, reducing pathogenic factors associated with PD so that neurodegeneration and secondary damage to neurons progresses at a slower rate. Therefore, BCG-induced neuroprotective immune responses will be beneficial in a slowly progressing disease, such as human PD.

In addition, a small protective effect may be clinically important because of a “threshold effect”—e.g., few symptoms may appear when 70% of dopaminergic function is lost, but severe symptoms may appear when 75% of dopaminergic function is lost. In this case, neuroprotection, or neurorestoration, of a small percent of dopaminergic function may be of great clinical value.

Further, neuroprotective vaccines may be used in combination with other therapeutic strategies, which may generate synergistic beneficial effects. Since currently there is no effective way to slow PD progression, new therapeutic approaches, even with a small beneficial effect, would be greatly welcomed, as long as they are completely safe—as is BCG vaccination.

Pilot studies on vaccination with CFA (alone) showed such vaccination is neuroprotective (data not shown). Our studies on BCG vaccination also showed it is neuroprotective (data not shown).

There has been recent interest in neuroprotective vaccines. So far neurprotective vaccines have focused on using Copaxone and no one has demonstrated neuroprotective effect with BCG vaccination before. BCG vaccination is very safe for humans, as billions of individuals have already been given this vaccine. Its protective effects come from being a general immune stimulant. Previous reviews of neuroprotective vaccines include: Schwartz M, Kipnis J., J Neurol Sci; 233(1-2):163-6 (2005) (Review); Schwartz M, Cell Mol Neurobiol. 21(6):617-27 (2001) (Review); and Schwartz M., J Mol Med., 78(11):594-7 (2001) (Review).

Previous published studies of neuroprotective vaccines that are suitable for human use administered Copaxone (glatiramer acetate). Copaxone is a FDA approved drug for treating multiple sclerosis. Copaxone is comprised of synthetic polypeptides of four amino acids in a random sequence, dissolved in an aqueous solution (without adjuvant). Because it contains many basic amino acids in random sequence, it is thought to induce T cell responses that cross-react with myelin basic protein (Schori, H., et al., Proc Natl Acad Sci USA 98:3398-3403 (2001); and Kipnis, J., et al., Trends Mol Med 8:319-323 (2002)). Copaxone injection is one of the few FDA approved treatments for MS. Without an adjuvant, Copaxone is weakly immunogenic and MS patients inject it daily subcutaneously. Importantly, almost all animal studies of Copaxone in neuroprotective vaccines used Copaxone emulsified in complete Freund's adjuvant (CFA), with the exception of two studies that found Copaxone (alone) was beneficial in mouse models of Alzheimer's disease (Frenkel, D., et al., J Clin Invest 115:2423-2433 (2005); and Butovsky, O., et al., Proc Natl Acad Sci USA 103:11784-11789 (2006)).

CFA contains inactivated Mycobacterium tuberculosis in mineral oil and is unsuited for human use. CFA is a strong immune stimulant, particularly of Th1-type CD4⁺ T cells (Yip, H. C., et al., J Immunol 162:3942-3949 (1999)). Many studies of neuroprotective vaccines did not report the effect of CFA (alone). Other studies reported that CFA (alone) had a neuroprotective effect that was not as great as CFA+antigen. For example, in the MPTP studies of Benner et al., control ovalbumin/CFA had some beneficial effect. Other studies, concluded that CFA itself was the neuroprotective factor (Jones, T. B., et al., J Neurosci 24:3752-3761 (2004)). Indeed, in the 6-OHDA rat model of PD, only CFA immunization was studied, and was found to be neuroprotective (Armentero, M. T., et al., Neurobiol Dis 24:492-505 (2006)).

Using the acute MPTP model, we found that vaccinating C57BL/6 mice with CFA, or Copaxone/CFA, had no protective effect. Using a chronic MPTP model, we found that vaccination with CFA with, or without, Copaxone, partially protected striatal dopamine (DA) and dopaine transporter (DAT). This suggests that a CFA-containing vaccine can be neuroprotective in a more slowly progressing degenerative process. In contrast, Copaxone (without adjuvant), Copaxone delivered in incomplete Freund's adjuvant (IFA, which is mineral oil without mycobacteria), and Copaxone delivered with alum, had no protective effect. Collectively, our data and those from previous reports, suggests that CFA is the major neuroprotective factor in these vaccines.

### Bacillus Calmette-Guerin (BCG) Vaccine is Neuroprotective.

Although previous reports highlighted the novel neuroprotective ability of vaccination with antigens in CFA in various animal models, CFA is not suitable for use in humans. However, the vaccine developed against pediatric tuberculosis meningitis and pulmonary tuberculosis contains Mycobacterium bovis that is closely related to the Mycobacterium tuberculosis that is in CFA. The vaccine was developed in the 1920s by Drs. Calmette and Guerin by passaging M. bovis until it had reduced virulence. Daughter strains were distributed throughout the world, and as a result of repeated passages under different conditions in different laboratories, the BCG vaccine strains diverged genetically, until lyophilization was introduced in the 1960s. There are now several different BCG vaccine strains, containing live attenuated BCG that is manufactured in different countries under GMP conditions.

The preparation and use of recombinant BCG vaccines is described in Horwitz, et al., PNAS, 97(25):13853-13858 (2000), the teaching of which is incorporated herein in its entirety by reference.

We believe that like CFA, BCG vaccination should be neuroprotective. We vaccinated C57BL/6 mice with a BCG vaccine (TheraCys®, Sanofi-Aventis) and observed an average 18% higher striatial DAT binding (P=0.027) and 16% higher DA levels (P=0.019) 21 days after chronic MPTP treatment in vaccinated vs. unvaccinated MPTP-treated mice.

### Mechanism(s) Underlying Neuroprotective Vaccines.

There is a continuing controversy as to whether Th1, Th2, Th3, or CD4⁺CD25⁺ T cells mediate the neuroprotection following vaccination in various animal models. For example, Dr. Schwartz and colleagues found that vaccines containing myelin basic protein (MBP), non-encephalomylitogenic peptides of MBP, or Copaxone in CFA were neuroprotective (Schori, H., et al., Proc Natl Acad Sci USA 98:3398-3403 (2001); Yip, H. C., et al., J Immunol 162:3942-3949 (1999); Moalem, G., et al., Nat Med 5:49-55 (1999); and Hauben, E., et al., J Clin Invest 108:591-599 (2001)). Since CFA is known to induce strong Th1-polarized immune responses (Yip, H. C., et al., J Immunol 162:3942-3949 (1999)), these studies suggest that vaccine-induced Th1 responses are the mediators of neuroprotection. Furthermore, the authors provided evidence that CD4⁺CD25⁺ regulatory T cell responses counteracted the vaccine-induced neuroprotective Th1 responses (Kipnis, J., et al., Proc Natl Acad Sci USA 99:15620-15625 (2002)). However, other reports from this group, as well as other groups, pointed to cytokines from Th2 and Th3 cells as being neuroprotective (Moalem, G., et al., J Autoimmun 15:331-345 (2000); Hofstetter, H. H., et al., J Neuroimmunol 134:25-34 (2003); Huang, D. W., et al., Neuron 24:639-647 (1999); and Hendrix, S., et al., J Neuroimmunol 184:100-112 (2007)). In opposition to the notion that regulatory T cells are deleterious for neuroprotection, the studies of Dr. Gendelman and colleagues suggest that CD4⁺CD25⁺ regulatory T cells mediate neuroprotection in their MPTP+Copaxone/CFA adoptive transfer model (Reynolds, A. D., et al., J Leukoc Biol 82:1083-1094 (2007)). Like CFA, BCG induces Th1-biased immune responses in rodents and humans (Teixeira, H. C., et al., Immunol Lett 46:15-19 (1995); and Kumar, M., et al., Immunology 97:515-521 (1999)). While this suggests that Th1 cells mediate the neuroprotection we observed following BCG vaccination, there is substantial evidence that inflammatory Th1 cytokines such as IFNγ and TNFα can also have regulatory properties (e.g., Tarrant, T. K., et al., J Exp Med 189:219-230 (1999); Manoury-Schwartz, B., J Immunol 158:5501-5506 (1997); Kim, E. Y., Immunol Lett 120:1-5 (2008); Kim, E. Y., et al., Arthritis Res Ther 10:R38 (2008); and Kim, E. Y., et al., Clin Immunol 127:98-106 (2008)). Activated T cells can also secrete BDNF, NGF, NT-3 and NT4/5 (see, e.g., Moalem, G., et al., J Autoimmun 15:331-345 (2000); Kerschensteiner, M., et al., J Exp Med 189:865-870 (1999); and Barouch, R., et al., J Neuroimmunol 103:112-121 (2000)). Such neurotrophins may help counteract intrinsic factors that promote DA degeneration, reduce the priming of surrounding neurons for secondary degeneration, and promote neurorestoration.

We envision several mechanisms, which are nonexclusive, that could contribute to the BCG-mediated neuroprotection/neurorestoration. These mechanisms, which shall not be read as being limiting, include:

1) The attenuated BCG slowly replicates (primarily in macrophages) of the recipient, inducing a long-term increase in the levels of circulating cytokines and chemokines, many of which can enter the CNS (Banks, W. A., et al., Neuroimmunomodulation 2:241-248 (1995)). These cytokines/chemokines limit microglia activation, and/or shift microglia toward a neuroprotective phenotype in the area of injury. Microglia distant from the injury stimulus should remain unactivated—for example, in the MPTP+Copaxone/CFA studies of Benner et al., microglia activation appeared limited to the SNc (Benner, E. J., et al., Proc Natl Acad Sci USA 101:9435-9440 (2004)).

2) BCG activates T cells which can enter CNS. At areas of CNS injury, “danger” signals cause T cells to release cytokines, neurotrophic factors, that beneficially affect microglia, astrocytes and/or neurons. Such protection would represent a nonantigen-specific bystander effect.

3) Possibly, but less likely, BCG (and CFA) activate T cells, some of which cross-react with self-antigens in the CNS. These activated T cells can recognize antigens in areas of injury that they were previously ignoring as naïve, unactivated T cells because 1) activated T cells have lower thresholds for performing effector functions than naïve cells and 2) there is increased antigen presentation in the area of injury because neurons upregulate expression of MHC class I after injury (Corriveau, R. A., et al., Neuron 21:505-520 (1998); Boulanger, L. M., et al., Curr Opin Neurobiol 11:568-578 (2001); and Boulanger, L. M., et al., Nat Rev Neurosci 5:521-531 (2004)), and because microglia take up antigens from damaged nerves and present them on their MHCl class II.

### Glutamate Decarboxylase/Glutamic Acid Decarboxylase

Glutamate decarboxylase or glutamic acid decarboxylase (GAD) is an enzyme that catalyzes the decarboxylation of glutamate to GABA and CO₂. GAD uses PLP as a cofactor. The biochemical reaction proceeds as follows:

HOOC—CH₂—CH₂—CH(NH₂)—COOH→CO₂+HOOC—CH₂—CH₂—CH₂NH₂

In mammals, GAD exists in two isoforms encoded by two different genes—Gad1 and Gad2. These isoforms are GAD₆₇ and GAD₆₅ with molecular weights of 67 and 65 kDa, respectively. GAD1 and GAD2 are expressed in the brain where GABA is used as a neurotransmitter, GAD2 is also expressed in the pancreas.

At least two more forms, GAD25 and GAD44 (embryonic; EGAD) are described in the developing brain. They are coded by the alternative transcripts of GAD1, I-80 and I-86: GAD25 is coded by both, GAD44—only by 1-80.

### Type I Diabetes

Both GAD₆₇ and GAD₆₅ are targets of autoantibodies in people who later develop type 1 diabetes mellitus or latent autoimmune diabetes. Injections with GAD₆₅ have been shown to preserve some insulin production for 30 months in humans with type 1 diabetes.

### Schizophrenia and Bipolar Disorder

Substantial dysregulation of GAD mRNA expression, coupled with downregulation of reelin, is observed in schizophrenia and bipolar disorder. The most pronounced downregulation of GAD67 was found in hippocampal stratum oriens layer in both disorders and in other layers and structures of hippocampus with varying degrees.

### GAD Peptide

As used herein, the term GAD peptide refers to a fragment of GAD. Some examples of GAD peptides are described in U.S. Pat. No. 6,011,139. The teaching in U.S. Pat. No. 6,011,139 is incorporated herein in its entirety by reference. Such teachings include, e.g., the identification of these peptides and methods of making these peptides.

### Other Agents

In some embodiments of the present invention, the composition (the vaccine composition and/or the neuroprotective drug described herein) can further include other agents or compounds that impart beneficial effects on a neurological disease.

Other agents that can be used with BCG to form the neuroprotective vaccine or neuroprotective drug disclosed herein can include, e.g., an agent that enhances BCG infection or a compound that activates the signaling pathways used by TLR-4, TLR-2, IFNgamma, IL2β, IL-6, IL-32 and IL-24. Such an agent can be, e.g., an immunostimulatory factor that simulates a typical host response to BCG infection. In some embodiments, the immunostimulatory factor is an inflammatory cytokine. In other embodiments, the immunostimulatory factor is one of IFNgamma, IL-2β, IL-6, IL-32, IL-24 and combinations thereof.

In some embodiments, the agent is a compound that simulates a BCG infection. In some embodiments, the compound binds to TLR-4 or TLR-2. In some other embodiments, the agent is selected from the group consisting of agonists of receptors binding to TLR-4 or TLR-2 and antibodies that activate these receptors.

In some embodiments, the agent is, e.g., a therapeutic substance effective for PD or an effective amount of a ligand to a group of receptors consisting of toll-like receptor-2 and toll-like receptor-4.

Other agents commonly in the art of vaccine and pharmaceuticals can also be included in the neuroprotective vaccine and neuroprotective drug described herein.

### Dosages

Dosages of the BCG and the various agents, therapeutic substances, and other agents are established in the art. Established dosages for BCG and other agents can be used, which is known to one of ordinary skill in the art.

### Formulation Carriers

The pharmaceutical composition described herein may be administered to a subject in need of treatment by a variety of routes of administration, including orally and parenterally, (e.g., intravenously, subcutaneously or intramedullary), intranasally, as a suppository or using a “flash” formulation, i.e., allowing the medication to dissolve in the mouth without the need to use water, topically, intradermally, subcutaneously and/or administration via mucosal routes in liquid or solid form. The pharmaceutical composition can be formulated into a variety of dosage forms, e.g., extract, pills, tablets, microparticles, capsules, oral liquid.

There may also be included as part of the pharmaceutical composition pharmaceutically compatible binding agents, and/or adjuvant materials. The active materials can also be mixed with other active materials including antibiotics, antifungals, other virucidals and immunostimulants which do not impair the desired action and/or supplement the desired action.

In some embodiments, the composition can be formulated into a formulation for bone, which can include a carrier such as collagen, atelocollagen (collagen treated to remove the immunogenic ends), hydroxyapatite, and a polymer, which is further described below. In these embodiments, the formulation can comprise a porous matrix or microspheres made of a polymeric material, which is further described below. In some embodiments, the polymer can be, e.g., polylactic acid or polylactide (PLA), or poly(lactic acid-co-glycolic acid), or another bioabsorbable polymer.

In one embodiment, the mode of administration of the pharmaceutical composition described herein is oral. Oral compositions generally include an inert diluent or an edible carrier. They may be enclosed in gelatin capsules or compressed into tablets. For the purpose of oral therapeutic administration, the aforesaid compounds may be incorporated with excipients and used in the form of tablets, troches, capsules, elixirs, suspensions, syrups, wafers, chewing gums and the like. Some variation in dosage will necessarily occur, however, depending on the condition of the subject being treated. These preparations should produce a serum concentration of active ingredient of from about 0.01 nM to 1,000,000 nM, e.g., from about 0.2 to 40 μM. A preferred concentration range is from 0.2 to 20 μM and most preferably about 1 to 10 μM. However, the concentration of active ingredient in the drug composition itself depends on bioavailability of the drug and other factors known to those of skill in the art.

In another embodiment, the mode of administration of the pharmaceutical compositions described herein is topical or mucosal administration. A specifically preferred mode of mucosal administration is administration via female genital tract. Another preferred mode of mucosal administration is rectal administration.

Various polymeric and/or non-polymeric materials can be used as adjuvants for enhancing mucoadhesiveness of the pharmaceutical composition disclosed herein. The polymeric material suitable as adjuvants can be natural or synthetic polymers. Representative natural polymers include, for example, starch, chitosan, collagen, sugar, gelatin, pectin, alginate, karya gum, methylcellulose, carboxymethylcellulose, methylethylcellulose, and hydroxypropylcellulose. Representative synthetic polymers include, for example, poly(acrylic acid), tragacanth, poly(methyl vinylether-co-maleic anhydride), poly(ethylene oxide), carbopol, poly(vinyl pyrrolidine), poly(ethylene glycol), poly(vinyl alcohol), poly(hydroxyethylmethylacrylate), and polycarbophil. Other bioadhesive materials available in the art of drug formulation can also be used (see, for example, Bioadhesion—Possibilities and Future Trends, Gurny and Junginger, eds., 1990).

It is to be noted that dosage values also varies with the specific severity of the disease condition to be alleviated. It is to be further understood that for any particular subject, specific dosage regimens should be adjusted to the individual need and the professional judgment of the person administering or supervising the administration of the aforesaid compositions. It is to be further understood that the concentration ranges set forth herein are exemplary only and they do not limit the scope or practice of the invention. The active ingredient may be administered at once, or may be divided into a number of smaller doses to be administered at varying intervals of time.

The formulation may contain the following ingredients: a binder such as microcrystalline cellulose, gum tragacanth or gelatin; an excipient such as starch or lactose, a disintegrating agent such as alginic acid, Primogel, corn starch and the like; a lubricant such as magnesium stearate or Sterotes; a glidant such as colloidal silicon dioxide; and a sweetening agent such as sucrose or saccharin or flavoring agent such as peppermint, methyl salicylate, or orange flavoring may be added. When the dosage unit form is a capsule, it may contain, in addition to material of the above type, a liquid carrier such as a fatty oil. Other dosage unit forms may contain other various materials which modify the physical form of the dosage unit, for example, as coatings. Thus tablets or pills may be coated with sugar, shellac, or other enteric coating agents. Materials used in preparing these various compositions should be pharmaceutically pure and non-toxic in the amounts used.

The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methylparabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

The pharmaceutical compositions of the present invention are prepared as formulations with pharmaceutically acceptable carriers. Preferred are those carriers that will protect the active compound against rapid elimination from the body, such as a controlled release formulation, including implants and microencapsulated delivery systems. Biodegradable, biocompatable polymers can be used, such as polyanhydrides, polyglycolic acid, collagen, and polylactic acid. Methods for preparation of such formulations can be readily performed by one skilled in the art.

Liposomal suspensions (including liposomes targeted to infected cells with monoclonal antibodies to viral antigens) are also preferred as pharmaceutically acceptable carriers. Methods for encapsulation or incorporation of compounds into liposomes are described by Cozzani, I.; Joni, G.; Bertoloni, G.; Milanesi, C.; Sicuro, T. Chem. Biol. Interact. 53, 131-143 (1985) and by Joni, G.; Tomio, L.; Reddi, E.; Rossi, E. Br. J. Cancer 48, 307-309 (1983). These may also be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

Other methods for encapsulating compounds within liposomes and targeting areas of the body are described by Sicuro, T.; Scarcelli, V.; Vigna, M. F.; Cozzani, I. Med. Biol. Environ. 15(1), 67-70 (1987) and Joni, G.; Reddi, E.; Cozzani, I.; Tomio, L. Br. J. Cancer, 53(5), 615-21 (1986).

The pharmaceutical composition described herein may be administered in single (e.g., once daily) or multiple doses or via constant infusion. The compounds of this invention may also be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The pharmaceutical compositions formed by combining the compounds of this invention and the pharmaceutically acceptable carriers, vehicles or diluents are then readily administered in a variety of dosage forms such as tablets, powders, lozenges, syrups, injectable solutions and the like. These pharmaceutical compositions can, if desired, contain additional ingredients such as flavorings, binders, excipients and the like according to a specific dosage form.

Thus, for example, for purposes of oral administration, tablets containing various excipients such as sodium citrate, calcium carbonate and/or calcium phosphate may be employed along with various disintegrants such as starch, alginic acid and/or certain complex silicates, together with binding agents such as polyvinylpyrrolidone, sucrose, gelatin and/or acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the active pharmaceutical agent therein may be combined with various sweetening or flavoring agents, coloring matter or dyes and, if desired, emulsifying or suspending agents, together with diluents such as water, ethanol, propylene glycol, glycerin and/or combinations thereof.

For parenteral administration, solutions of the compounds of this invention in sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of a compound of this invention. Capsules and cartridges (made, for example, from gelatin) for use in an inhaler or insufflator may be formulated containing a powder mix of a compound or compounds of the invention and a suitable powder base such as lactose or starch.

The pharmaceutical composition described herein can be formulated alone or together with the other agent in a single dosage form or in a separate dosage form. Methods of preparing various pharmaceutical formulations with a certain amount of active ingredient are known, or will be apparent in light of this disclosure, to those skilled in this art. For examples of methods of preparing pharmaceutical formulations, see Remington's Pharmaceutical Sciences, Mack Publishing Company, Easton, Pa., 19th Edition (1995).

In some embodiments, the composition of the various embodiments disclosed above can be formulated into implants, scaffolds, patches, etc.

### Method of Using the Composition

Generally, the vaccine or drug composition of invention can be administered to a subject (e.g., a human being) to treat, prevent, or ameliorate a neurological disease or an autoimmune disease. In some embodiments, the neurological disease is one of PD, Alzheimer's disease, and amyotrophic lateral sclerosis. In some embodiments, the neurological disease is a disease or disorder related to neuro-autoimmunity.

The autoimmune disease can be any disease or disorder related to autoimmunity. Autoimmune diseases or disorder are documented and known in the art. Examples of autoimmune diseases include, but are not limited to, systemic lupus erythematosus, Sjogren syndrome, Hashimoto thyroiditis, rheumatoid arthritis, type 1 diabetes, polymyositis, scleroderma, Addison disease, vitiligo, pernicious anemia, glomerulonephritis, and pulmonary fibrosis. In some embodiments, the autoimmune disease is rheumatoid arthritis or type 1 diabetes.

### EXAMPLES

The invention is described in more detail in the following illustrative examples. Although the examples can represent only selected embodiments of the invention, it should be understood that the following examples are illustrative and not limiting.

### Example 1

### Studies on Neuroprotective Vaccine for PD

### Abstract:

Vaccination with CNS proteins in adjuvants can induce immune responses that inhibit neuronal degeneration in neurodegeneration. We evaluated whether directing immune responses to the CNS may preserve dopaminergic neurons in the 1-Methyl-4-Phenyl-1,2,3,6-Tetrahydro-Pyridine (MPTP) mouse model of Parkinson's disease (PD). We found that vaccination with complete Freund's adjuvant (CFA) alone, or with Copaxone® in CFA, conferred a neuroprotective effect on the vaccinated mice as reflected by increased striatal dopamine transporter (DAT) binding. Stereological analysis revealed no difference in the number of substantia nigra pars compacta (SNc) tyrosine hydroxylase positive (TH+) cells. Bacillus Calmette-Guerin (BCG) vaccine against tuberculosis contains mycobacteria closely resembling the active component of CFA, and is safe for human use. We tested whether BCG vaccination can be neuroprotective in the MPTP mouse model. Mice that were pre-vaccinated with BCG had 18% higher DAT (P<0.01) and 17% higher dopamine (DA) levels (P<0.05) than unvaccinated mice after MPTP. We did not observe differences in the number of SNc TH+ cells, but the density of microglia in SNc in BCG-vaccinated mice was reduced compared to unvaccinated MPTP-treated mice (p<0.05) and similar to that in mice that did not receive MPTP. We surmise that BCG-activated immune responses cross the blood brain barrier and exert neuroprotective effects. Thus, a vaccine safe for human use can induce immune responses that preserve dopaminergic neuron function in a mouse model of PD. These findings may lead to new approaches to slow the progression of human PD and other neurodegenerative disorders.

### Methods:

Copaxone® Vaccination:

Male C57B16 mice (8-10 weeks old) were immunized with CFA (1 mg/ml mycobacteria) alone or Copaxone® (100 ug) in CFA at the base of tail. 10 days later, the mice were treated with MPTP (20 mg/kg i.p. per day for 5 consecutive days).

BCG Vaccination:

Mice were immunized intraperitoneally with 2×10⁷ CFU BCG (Strain Tice or Connaught, Sanofi Pasteur) diluted in 200 ul diluent. 10 days later, mice were treated with MPTP.

DA and DAT Assays:

Mice were sacrificed 7 or 21 days after MPTP injection, and the striatum was harvested by microdissection, while the caudal halves of the brains were fixed in 4% PFA for nigra TH+ cell counting. Striatal DA was measured by HPLC and striatal DAT was evaluated by [³H]WIN-35,428 binding assay.

Immunohistochemistry and Stereology:

Dopaminergic neurons in the SNc were visualized by TH immunostaining and TH+ cells were quantified in SNc by stereological analysis (Stereo Investigator 8.0, MBF Bioscience). For microglia quantification, animals were sacrificed 3 days after MPTP injection and were transcardially perfused with 4% PFA, followed by overnight post-fixation in PFA. Brains were then cryopreserved and sectioned into 30 um slices for Iba-1 (a microglia marker) staining SNc Iba-1+ cell density was quantified by stereological analysis.

### Results:

### Establishing a Subacute MPTP Model

A number of treatments can temporarily ameliorate PD symptoms, but none can slow the progressive loss of dopaminergic neurons. A growing number of studies have shown that vaccination with CNS proteins can induce immune responses that inhibit neuronal degeneration in different animal models of neuronal injury and neurodegeneration (Moalem G, et al., Nat Med 5:49-55 (1999); Angelov D N, et al, Proc Natl Acad Sci USA 100:4790-4795 (2003); and Kipnis J, et al., J Neurotrauma 20:559-569 (2003)). Our initial studies using an acute MPTP model (15 mg/kg for 4× at 2 h intervals) found no consistent neuroprotection conferred by Copaxone®/CFA vaccination. We surmised that high-dose MPTP administration causes a rapid loss of dopaminergic neurons that cannot be limited by vaccine-induced immune responses. We therefore tested if vaccination could be neuroprotective in a subacute MPTP model of PD.

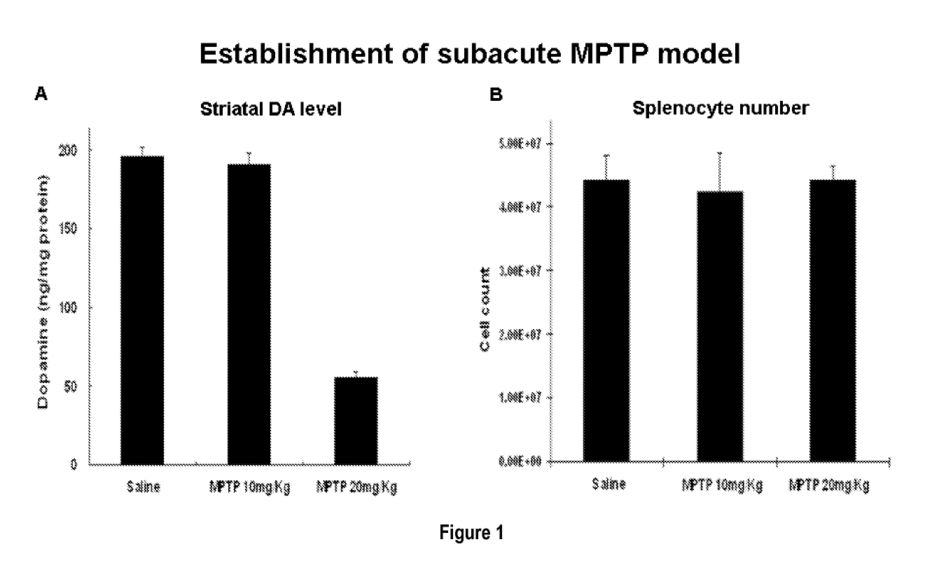

To establish a subacute MPTP model, we tested administration of 10 mg/kg/day and 20 mg/kg/day MPTP for consecutive 5 days. We found that the 20 mg dose decreased striatal dopamine level by 70% compared to PBS-treated control mice (FIG. 1A). Neither doses of MPTP significantly altered splenocytes number, suggesting that these MPTP doses had little immuno-toxicity (FIG. 1B).

FIG. 1 shows the characterization of a subacute MPTP model. (A) Four days after the last treatment, 20 mg/kg/day MPTP for 5 days decreased striatal dopamine level by 70% compared to PBS treated mice, while 10 mg/kg/day had no obvious effect on striatal dopamine levels. (B) Subacute MPTP treatment did not affect splenocyte counts of recipient mice, suggesting little/no immunotoxicity. Data are represented as mean±SEM.

### 2. Copaxone®/CFA Vaccination in the Subacute MPTP Model

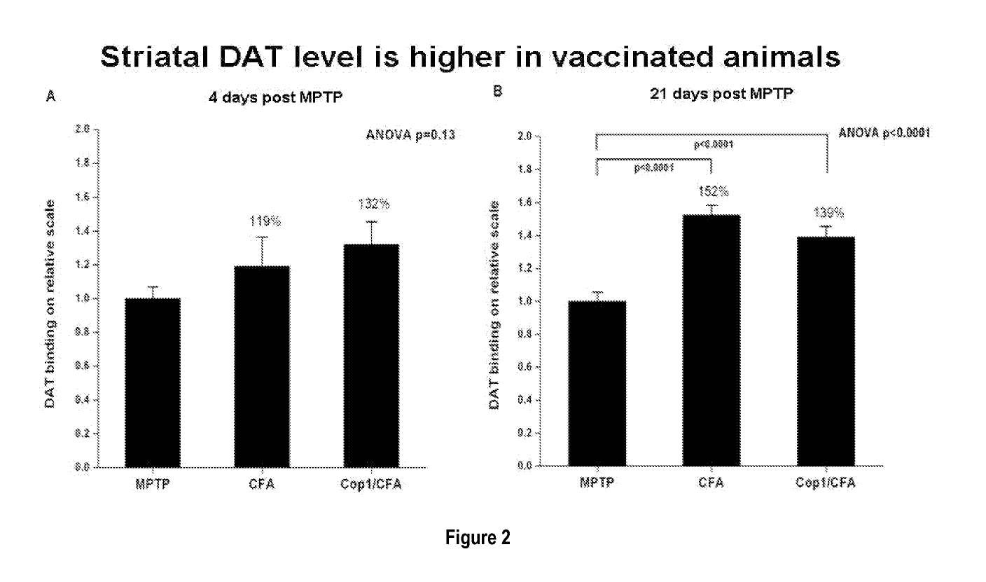

Mice were vaccinated with Copaxone®/CFA (100 μg in 1 mg/ml CFA) 10 days prior to MPTP treatment. Non-vaccinated mice and mice vaccinated with CFA (alone) served as controls. Animals were sacrificed at 4 and 21 days after subacute MPTP treatment, and their striatal dopamine and DAT levels were examined. The two time points were chosen because we were interested in evaluating both neuroprotective and neuro-restorative effects by vaccination. [³H]WIN-35,428 binding assay revealed elevated striatal DAT binding in the striata of both Copaxone®/CFA and CFA (alone) vaccinated animals: 4 days post-MPTP, CFA and Copaxone®/CFA groups had higher DAT binding capacity (19% and 32% respectively, FIG. 2A) compared to controls. Twenty one days post-MPTP, both Copaxone®/CFA and CFA (alone) had higher DAT binding (52% and 39% respectively, p<0.0001 for both comparisons, FIG. 2B).

Striatal dopamine levels were slightly higher in vaccinated groups than in non-vaccinated controls at both time points examined, although these differences were not statistically significant (data not shown). Stereological analysis on brains from MPTP and Copaxone®/CFA groups found similar nigral TH+ cell counts (Copaxone®/CFA:MPTP=7636±328:7537±225). Therefore protection of striatal nerve fibers was not due to better preservation of SNc dopaminergic neuron bodies.

Our results using the subacute MPTP model indicate that CFA (alone) vaccination can protect terminals in the striatum and/or enhance the residual capacity of the remaining dopaminergic cells to maintain dopaminergic function.

FIG. 2 shows that CFA and Copaxone®/CFA vaccination led to greater striatal DAT preservation and improved recovery. A). [³H]WIN-35,428 binding assays showed that both CFA and Copaxone®/CFA vaccinated animals had higher (but non-significant) DAT levels than non-vaccinated mice 4 days post MPTP. B). both CFA and Copaxone®/CFA vaccinated animals had higher DAT levels than non-vaccinated mice 21 days post MPTP (p<0.0001 by t-test for either group vs. MPTP control). Results from two studies were combined. Data shown are mean±SEM.

### Effects of BCG Vaccination in the Subacute MPTP Model.

The finding that CFA (alone) vaccination was neuroprotective suggested that CFA-induced immune responses, and not Copaxone, are the major factors underlying the vaccines' beneficial effects. CFA contains heat inactivated Mycobacterium tuberculosis in mineral oil and is unsuited for human use. However, a vaccine containing a closely related live mycobacterium, Bacillus Calmette-Guerin (BCG), is in wide clinical use to prevent tuberculosis. We concluded that BCG vaccination should also be neuroprotective.

### 1). BCG Vaccination Protects Striatal DAT and DA

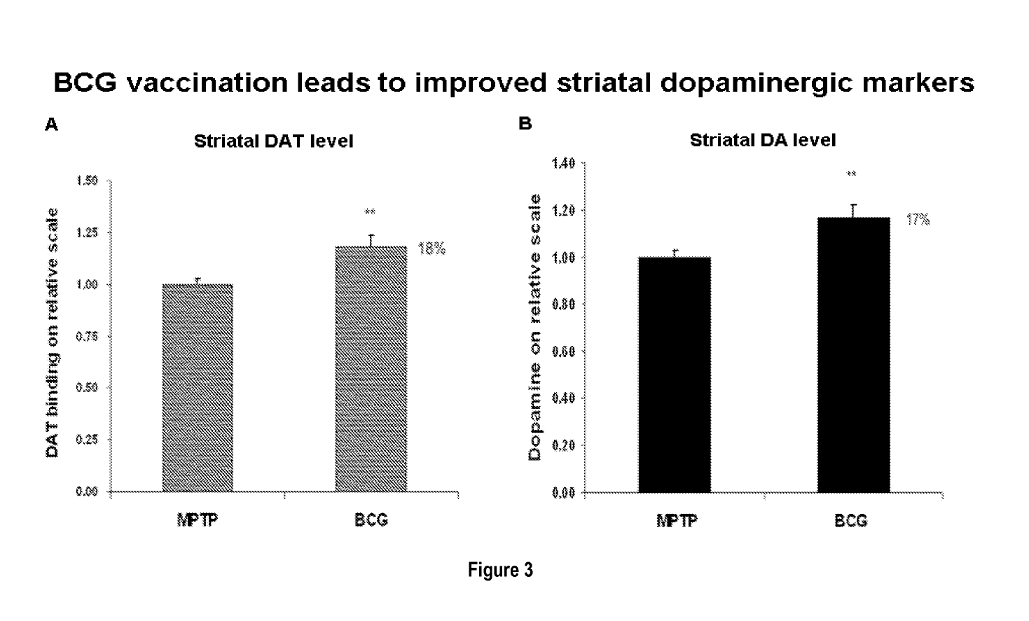

Mice were vaccinated with 2×10⁷ CFU BCG i.p. 10 days before subacute MPTP treatment. A group of non-vaccinated mice received MPTP and served as controls. Twenty one days after MPTP treatment we found 18% higher DAT levels (P<0.01, FIG. 3A) and 17% higher dopamine levels (P=0.01, FIG. 3B) in BCG vaccinated mice compared to non-vaccinated MPTP-treated mice. Stereological analysis found similar nigral TH+ cell counts in both groups (BCG:MPTP=6400±424:6421±477).

FIG. 3 shows that BCG conferred neuroprotection to vaccinated mice after subacute MPTP administration. A). Striatal tissues from BCG-vaccinated mice (n=18) showed 18% higher [3H]WIN-35,428 binding (p<0.01 by two-tailed t-test) than non-vaccinated MPTP-treated control mice (n=17). B). Striatal DA levels were 17% higher (p=0.01 by two-tailed t-test) in BCG-vaccinated mice (n=16) than in control mice (n=17). Results from two separate experiments were pooled. Data shown are mean±SEM.

### 2). BCG Vaccination Inhibits MPTP-Induced Increase in SNc Microglia

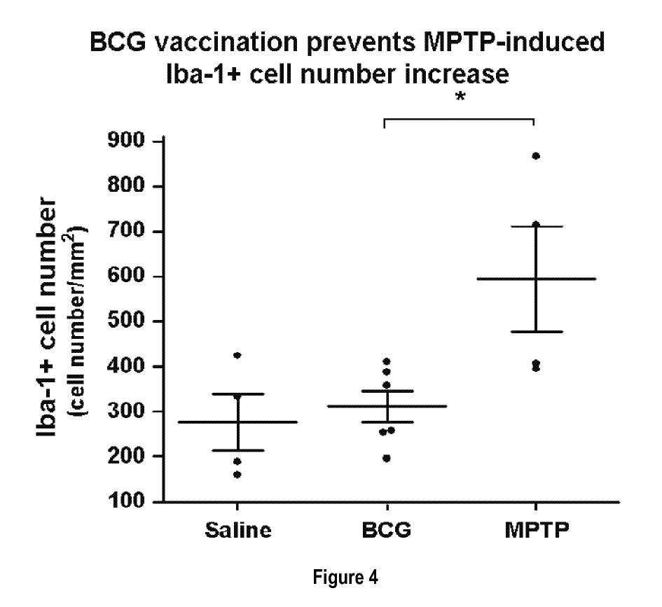

Microglia are the resident immune cells in the CNS. Previous studies suggested that microglia play an important role in MPTP induced nigro-striatal system damage, while blockade of microglia activation was neuroprotective (Wu D C, et al., J Neurosci 22:1763-1771 (2002); Kohutnicka M, et al., Immunopharmacology 39:167-180 (1998); and Czlonkowska A, et al: Neurodegeneration 5:137-143 (1996)). MPTP intoxication activates microglia in SNc, and their number peaks around 3 days after MPTP (Kohutnicka M, et al., Immunopharmacology 39:167-180 (1998)). Stereological counting of SNc Iba-1+ (a microglia marker) cells from experimental and control mice 3 days post-MPTP revealed that BCG-vaccinated mice had reduced number of Iba-1+ cells compared to unvaccinated MPTP-treated mice (FIG. 4, p<0.05), indicating that BCG vaccination reduces the number of MPTP-induced microglia in the SNc.

FIG. 4 shows average SNc Iba-1+ cell number from BCG vaccinated mice is lower than that from MPTP control mice. Unvaccinated MPTP-treated mice had the expected increase in SNc Iba-1+ cell number, while that from BCG-vaccinated mice remained similar to saline controls. Difference between BCG and saline groups was statistically significant (*: p<0.05). Dots represent individual mice. Data shown is group average±SEM.

### Discussion

We evaluated whether directing immune responses to the CNS may provide a novel treatment strategy for preserving dopaminergic neurons in the MPTP mouse model of PD. We established a subacute MPTP model to test this vaccination strategy. We observed that vaccination with Copaxone®/CFA or CFA (alone) preserved striatal DAT without preserving TH+ cells in SNc. It is likely that immunization protected terminals in the striatum and/or enhanced the residual capacity of the remaining dopaminergic cells to maintain dopaminergic function.

Based on our finding that a general immuno-stimulation with CFA (alone) was as neuroprotective as Copaxone®/CFA vaccination, we conclude that BCG vaccination can also be neuroprotective. BCG vaccination significantly protected DA and DAT in the striatum and inhibited the MPTP-induced increase in SNc microglia density. There could be multiple nonexclusive mechanisms underlying BCG's neuroprotective effects:

1) BCG slowly replicates and causes a long-term increase in the levels of circulating immune factors (e.g., cytokines, chemokines or other factors), many of which can enter the CNS. These immune factors may have a beneficial effect on glia, such as limiting influx of microglia precursor cells, microglia proliferation, microglia activation, or shifting microglia phenotype toward a neuroprotective phenotype in the area of injury. Alternatively, these factors may have a neuroprotective effect on neurons in the area of injury.

2) BCG activates T cells and antigen presenting cells (APC, such as macrophages and dendritic cells) which can enter CNS. At areas of CNS injury, “danger” signals cause T cells and/or APC to release factors that beneficially affect glia and/or neurons. Such protection would be a nonantigen-specific bystander effect. BCG infection may lead to activation of toll-like receptors (e.g., TLR-4 and TLR-2), which exret a neuroprotective effect.

3) Less possibly, BCG may activate some T cells that cross-react with CNS self-antigens. Activated T cells can recognize antigens in areas of injury that they were previously ignoring as naïve, unactivated T cells because 1) activated T cells have lower thresholds for performing effector functions than naïve cells and 2) there is increased antigen presentation in the area of injury because neurons can upregulate expression of MHC class I after injury, and because microglia take up antigens from damaged nerves and present them on their MHC class II.

### Conclusions:

Previous neuroprotective vaccines aimed to induce T cell responses to brain antigens (e.g., by immunizing with myelin basic protein or Copaxone). Here, we show that BCG vaccination, which does not induce immune responses to specific CNS antigens, elicits responses that preserve dopaminergic function in a mouse model of PD. By extension, BCG vaccination may also be protective in other neurodegenerative disorders, such as Alzheimer's disease. The BCG vaccine has been given to billions of individuals. Vaccination with BCG, or pharmacological modulation of the pathways induced by BCG infection (cytokines, cytokine receptors, TLR-4, TLR-2 and their signaling pathways (e.g., JAK-STAT, MyD88, TRAF6)) may represent new modalities to slow disease progression in human PD as well as other neurodegenerative disorders.

### Example 2

### Studies on Effects of BCG Vaccination on Dopamine Level

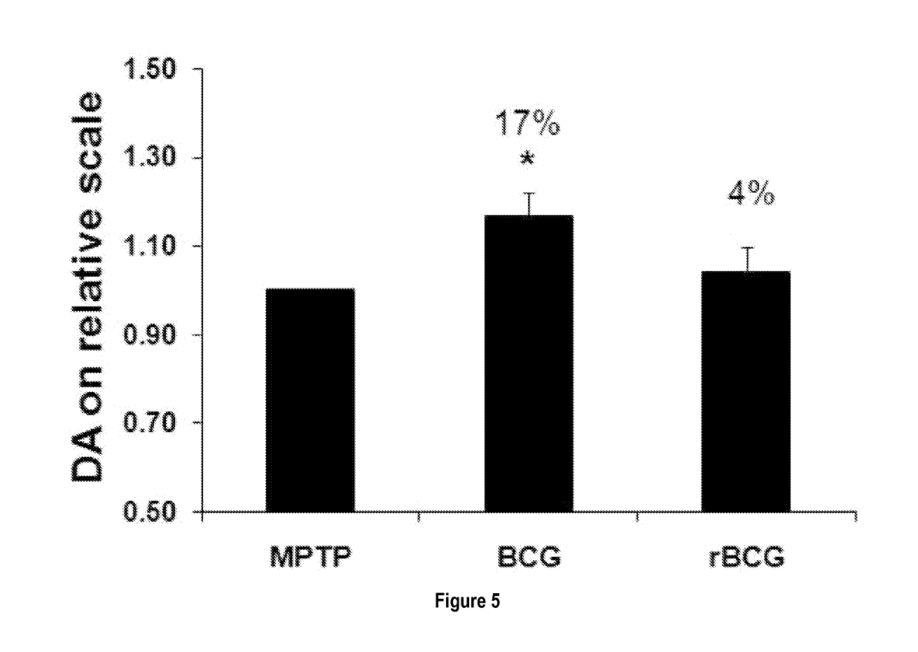

Studies were performed to test the effects of BCG vaccination on DA level. The results are shown in FIG. 5. FIG. 5 shows that relative to the control, BCG vaccination results in 17% of enhancement on the DA level, and recombinant BCG vaccination (rBCG) results 4% of enhancement on the DA level.

### Example 3

### Studies on Effects of BCG Vaccination on WIN35,428 Binding

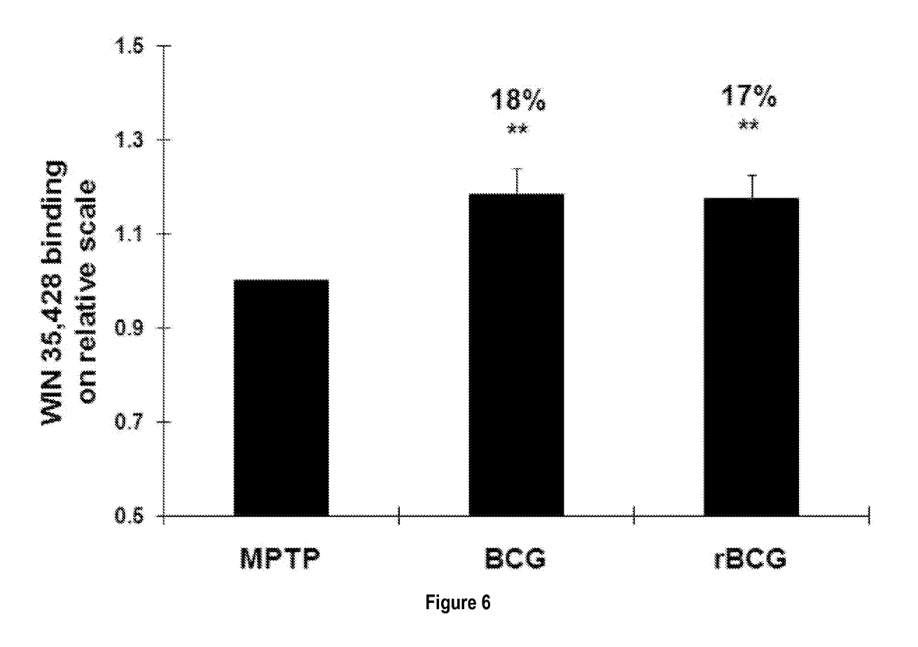

Studies were performed to test the effects of BCG vaccination WIN35,428 binding. The results are shown in FIG. 6. FIG. 6 shows that relative to the control, BCG vaccination results in a 18% enhancement on WIN35,428 binding, and recombinant BCG vaccination (rBCG) results in a 17% of enhancement on WIN35,428 binding.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A composition, which is a vaccine composition or a pharmaceutical composition, comprising an effective amount of Bacillus Calmette-Guerin (BCG) and an optional second agent, wherein the composition is effective for inhibiting the progress of a neurological disease or causing a neurological disease to progress at a slower rate.

2. The composition of claim 1, wherein the second agent is effective for Parkinson's disease (PD).

3. The composition of claim 1, wherein the second agent is an enzyme, a peptide, a compound or a combination of these.

4. The composition of claim 3, wherein the enzyme is glutamic acid decarboxylase 65 (GAD65), glutamic acid decarboxylase 67 (GAD67), and wherein the peptide is a GAD peptide.

5. The composition of claim 1, wherein the neurological disease is selected from the group consisting of PD, Alzheimer's disease, and amyotrophic lateral sclerosis.

6. The composition of claim 1, further comprising an adjuvant.

7. The composition of claim 1, comprising the second agent and further an excipient,
wherein the composition comprises:
a formulation comprising the BCG; and
a formulation comprising the second agent.

8. The composition of claim 7, wherein the excipient comprises a pharmaceutically acceptable carrier.

9. The composition of claim 1 in a formulation for systemic delivery or local delivery.

10. The composition of claim 7, wherein the formulation comprising the second agent is a formulation for fast release or sustained release.

11. The composition of claim 8, wherein the formulation comprising the second agent is a formulation for fast release or sustained release.

12. The composition of claim 1 in a formulation for oral delivery, inhalation, injection, implant, topical delivery, or transdermal delivery.

13. The composition of claim 1, comprising the second agent, the second agent comprising an effective amount of an immunostimulatory factor that simulates a typical host response to BCG infection.

14. The composition of claim 13, wherein the immunostimulatory factor is an inflammatory cytokine.

15. The composition of claim 13, wherein the immunostimulatory factor is selected from the group consisting of IFNgamma, IL2β, IL-6, IL-32, IL-24 and combinations thereof.

16. The composition of claim 1, comprising the second agent, the second agent comprising an effective amount of a compound that simulates a BCG infection.

17. The composition of claim 17, wherein the compound binds to TLR-4 or TLR-2.

18. The composition of claim 17, wherein the compound is selected from the group consisting of agonists of receptors binding to TLR-4 or TLR-2 and antibodies that activate these receptors.

19. The composition of claim 1, comprising the second agent, the second agent comprising an effective amount of a compound that activates the signaling pathways used by TLR-4, TLR-2, IFNgamma, IL2β, IL-6, IL-32 and IL-24.

20. A method, comprising forming a composition according to claim 1.

21. A method of treating, preventing, or ameliorating a neurological disease or an autoimmune disease, comprising:
administering to a subject a composition according to claim 1.

22. The method according to claim 21, wherein the neurological disease is selected from the group consisting of PD, Alzheimer's disease, and amyotrophic lateral sclerosis.
